(19) United States
(12) Patent Application Publication
(10) Pub. No.: US 2022/0415514 A1
(43) Pub. Date: Dec. 29, 2022

(54) ASYMPTOMATIC COMPLEX DISEASE MONITORING ENGINE

(71) Applicant: International Business Machines Corporation, Armonk, NY (US)

(72) Inventors: VIBHA Anand, Cambridge, MA (US); Bum Chul Kwon, Cambridge, MA (US); MOHAMED GHALWASH, Yorktown Heights, NY (US); Kenney Ng, Arlington, VA (US)

(21) Appl. No.: 17/360,617

(22) Filed: Jun. 28, 2021

(51) Int. Cl. G16H 50/30; G16H 50/50; G16H 10/60; G16H 50/70; G16H 50/20; G06N 7/00

(52) U.S. Cl. CPC G16H 50/30; G16H 50/50; G16H 10/60; G16H 50/70; G16H 50/20; G06N 7/005; G16H 10/40

(57) ABSTRACT

A processor may receive data associated with one or more users regarding biomarkers for a condition. The processor may determine one or more progression trajectories from the data using progression modeling. The processor may identify one or more granular stages associated with the one or more progression trajectories. The processor may generate a risk assessment for development of the condition associated with the one or more progression trajectories and the one or more granular stages.

### BACKGROUND

The present disclosure relates generally to the field of disease monitoring, and more specifically to determining a risk for development of a disease in an asymptomatic individual based on individualized disease trajectory predictions.

The asymptomatic period for Type 1 diabetes may last from several months to years and provides an opportunity for screening, monitoring, and possible preventive interventions at the individual or population level. By measuring sensitive and specific islet autoantibody (types) titer levels and characterizing them as positive or negative throughout an individual's life course in routine practice (often at discrete intervals), it has been shown that distinct trajectories of islet autoimmunity exist.

### SUMMARY

Embodiments of the present disclosure include a method, computer program product, and system for determining an arrangement of edge computing devices configured to meet the edge computing needs of a user.

A processor may receive data associated with one or more users regarding biomarkers for a condition. The processor may determine one or more progression trajectories from the data using progression modeling. The processor may identify one or more granular stages associated with the one or more progression trajectories. The processor may generate a risk assessment for development of the condition associated with the one or more progression trajectories and the one or more granular stages.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a system diagram of an exemplary system for individualized disease trajectory predictions, in accordance with aspects of the present disclosure.

FIG. 2 is a flowchart of an exemplary method for individualized disease trajectory predictions, in accordance with aspects of the present disclosure.

FIG. 3A illustrates a cloud computing environment, in accordance with aspects of the present disclosure.

FIG. 3B illustrates abstraction model layers, in accordance with aspects of the present disclosure.

FIG. 4 illustrates a high-level block diagram of an example computer system that may be used in implementing one or more of the methods, tools, and modules, and any related functions, described herein, in accordance with aspects of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of disease monitoring, and more specifically to determining a risk for development of a disease in an asymptomatic individual based on individualized disease trajectory predictions. While the present disclosure is not necessarily limited to such applications, various aspects of the disclosure may be appreciated through a discussion of various examples using this context.

The asymptomatic period for Type 1 diabetes may last from several months to years and provides an opportunity for screening, monitoring, and possible preventive interventions at the individual or population level. By measuring sensitive and specific islet autoantibody (types) titer levels and characterizing them as positive or negative throughout an individual's life course in routine practice (often at discrete intervals), it has been shown that distinct trajectories of islet autoimmunity exist. The present disclosure relates to personalized risk assessment application involving matching an individual's trajectory to known islet autoimmunity trajectories and assessing his/her risk for developing Type 1 diabetes.

In some embodiments, a processor may receive data associated with one or more users regarding biomarkers for a condition. In some embodiments, the data may be longitudinal data from clinical research into the underlying pathophysiology of the disease process for the condition. In some embodiments, the data may be obtained from patients at varying times and stages of disease progression. In some embodiments, the data may be regarding physiological or biological characteristics that mark disease progress. In some embodiments, the data may be the available data from research on the condition, including from disease registries, observational studies, electronic health records, etc. In some embodiments, the data may relate to the presence or absence of biomarkers detected in blood-serum (or variations in biomarkers or amounts) that clinicians believe is indicative of progression of the condition and different stages of the condition.

In some embodiments, the data may be regarding heterogeneously progressing islet auto immunity in those at risk of developing Type 1 diabetes. For example, the islet autoantibodies may be measured in the blood serum of individuals with genetic or familial high risk for Type 1 diabetes. In some embodiments, the islet autoantibodies may be measured starting at an early age and then at discrete intervals from there onwards.

In some embodiments, the processor may determine one or more progression trajectories from the data using progression modeling. In some embodiments, the progression trajectories may describe the heterogeneity of progression of the condition across populations. In some embodiments, the progression trajectories may be obtained from disease progression modeling that quantitatively characterizes the course of disease progression from patients' data contained in longitudinal health records. In some embodiments, the progression modeling (e.g., disease progression modeling) may utilize algorithms or models including (but not limited to) systems biology models, data-driven models, semi-mechanistic models, path models, tree-based models, hierarchical latent variable models, Gaussian process models, Bayesian networks, and state-space models. In some embodiments, the disease progression modeling may be based on an understanding of the output probabilities of diverse features for individual states. In some embodiments, the disease progression modeling may be based on differences in state transition patterns between patients. In some embodiments, the disease progression modeling may be based on an association among state transition patterns and patient outcome variables (e.g., disease onset, death, discharge, etc.).

In some embodiments, the processor may determine one or more progression trajectories using machine learning that utilizes a Hidden Markov Model (“HMM”). In some embodiments, the HMM may represent the progression of a subject through disease stages as transitions between hidden states, or latent states. In some embodiments, the hidden states, when learned from patient data, may characterize a set of probability distributions of multiple observed measures. In some embodiments, the state transitions (among latent states) may be represented using a transition matrix. In some embodiments, the transition matrix may define the probability of transitioning between hidden states and may be constrained based on domain knowledge (e.g., knowledge obtained about the disease by subject matter experts) or pragmatic assumptions (e.g., chronic conditions exhibit worsening symptoms as time progresses). In some embodiments, the target disease may be represented by a fixed number of typical disease states, the progression may be characterized by a Markov process, and the observed measures may be the manifestations of underlying disease states. In some embodiments, the HMI may characterize the progression of the disease as a series of transitions between typical disease states, providing an interpretable summary of disease progression.

HMM models exhibit several desirable properties that make them well suited for disease progression modeling. First, they are probabilistic in nature and are able to represent uncertainties in clinical data organically. Second, they are able to handle missing data, a common issue in observational studies. Third, users can apply their domain knowledge into modeling disease progression dynamics by setting various constraints. For example, users can prohibit a model from stepping backward in diseased states when chronic diseases are involved, as the damage caused by the disease is irreversible and affected areas gradually lose their functions, and backward-enabled progression, where patients can recover from the diseases. Fourth, HMMs are unsupervised models that do not require a particular outcome task. Fifth, HMMs provide interpretability by a small number of ‘states’ that are associated with multiple observed variables.

In some embodiments, the one or more progression trajectories may be assessed using visual analytics methods. In some embodiments, the processor may utilize visual analytics methods to explore and discover patterns from longitudinal data in clinical studies. In some embodiments, the processor may utilize visual analytic methods to represent temporal event sequences. In some embodiments, visual analytic methods may allow users (e.g., clinicians) to steer the algorithm by user interactions, so that users can find meaningful summaries of event sequences. Visualization analytic methods may allow users to directly interact with visual representations in order to perform various queries on temporal event sequences, such as filtering event sequences by length or an attribute. By doing so, users do not need to write complex queries in order to get answers to their analytic questions that are now being answered by real-time interactions.

In some embodiments, DPVis, an interactive visualization tool developed for users to explore disease progression patterns, may be utilized. In some embodiments, the DPVis tool may integrate state-space models such as HMM models for exploring disease progression patterns from longitudinal observational data. In some embodiments, the DPVis may be a visual analytics system that consists of multiple, coordinated views, where each view provides a summary and details of the HMI states and the state transition patterns. In some embodiments, the visualization may help users examine the summary of latent states by inspecting the probabilities of input variables per state. In some embodiments, the users may also evaluate the probabilities of other associated variables (per state) that were unused in the modeling process. In some embodiments, the users may summarize the discovered longitudinal clusters with respect to state transitions, i.e., trajectories. In some embodiments, the users may find the volume of individuals and compare heterogeneity in terms of progression states by viewing these trajectories. In some embodiments, the users may also further group individuals based on state transition patterns. In some embodiments, the state transition patterns may show the age when transitions occur and how long patients spend in a state (the duration or dwell time).

In some embodiments, the processor may identify one or more granular stages associated with the one or more progression trajectories. In some embodiments, the granular stages may be endotypes of the disease that are subtypes of the disease defined by a distinct pathophysiological mechanism. In some embodiments, presymptomatic endotypes (e.g., subgroups from patients before they exhibit symptoms associated with an active disease state) may be defined based on age, or baseline variables such as sex, human leukocyte antigen characteristics, family medical history, etc. In some embodiments, the endotypes may be ascertained by probabilistic modeling of the disease. In some embodiments, endotypes may be groups of latent states discovered by probabilistic models. As an example, the states may be age-dependent states. In some embodiments, one or more endotypes may be ascertained from the disease progression trajectory by modeling dwell times in a disease stage, clinical age, and covariates (e.g., baseline features such as gender, human leukocyte antigen characteristics, family medical history). For example, an endotype may be ascertained from a function of model states, dwell times in a state, and covariates. In some embodiments, an endotype may be ascertained from information related to how many individuals are observed to enter or exit a state at a certain median age. In some embodiments, how long individuals stay in a certain state may be indicative of a pathophysiological state. In some embodiments, data regarding states may be used as a covariate with other features (e.g., human leukocyte antigen characteristics, family medical history, etc.) to divide the data on states and trajectories into endotypes.

In some embodiments, endotypes may be ascertained by analyzing disease trajectories learned by disease progression models using human in the loop visual analytics systems such as DPVis. In some embodiments, DPVis may reveal endotypes by characterizing disease trajectories. In some embodiments, the disease trajectories may be represented as sequences of states discovered by HMIs. In some embodiments, the visualizations may show what sequences of states are included per trajectory and how the states are manifested by different biomarkers at different ages, thus characterizing the pathophysiology of the disease process. In some embodiments, the visualizations may reveal how many participants belong to each trajectory or sub-trajectory. In some embodiments, the DPVis may allow users to query sub-trajectories by filtering for patients with different biomarkers, timing of state transitions, health outcomes, age groupings, etc.

In some embodiments, the processor may generate a risk assessment for development of the condition associated with the one or more progression trajectories and the one or more granular stages. In some embodiments, the risk assessment generated may be for a particular granular stage combined with a particular disease progression trajectory. In some embodiments, a risk assessment may be generated for each combination of granular stage (e.g., one granular stage of the one or more granular stages) and progression trajectory (e.g., one progression trajectory of the one or more progression trajectories). In some embodiments, the risk assessment may be a calculation of the likelihood of (for a person in the particular granular stage having the particular disease progression trajectory) developing the disease by a certain age. In some embodiments, the risk assessments may calculate the odds of developing the disease by multiple ages (e.g., the odds of developing the disease by age 10, 15, 20, etc.).

In some embodiments, generating the risk assessment may involve generating a map plotting granular stages (e.g., endotypes) and respective disease progression trajectories. Based on where an individual lies on the plot of endotype and disease progression trajectories, a risk for developing the disease may be calculated for that individual.

In some embodiments, the risk may be calculated using risk calculation methods from the data associated with one or more users regarding biomarkers for a condition. In some embodiments, the data regarding biomarkers for the condition may be grouped based on granular stage and disease progression trajectory to determine the risk calculation. In some embodiments, the risk may be calculated using statistical modeling (e.g., linear regression, logistic regression, Cox model, etc.), machine learning modeling (e.g., Random Forest, Support Vector Machines, etc.), or deep learning modeling (e.g., neural networks) that may be derived from/trained on data for that endotype and then used later to estimate the risk for a new patient.

In some embodiments, the processor may receive new user data regarding biomarkers for the condition. The new patient data may include data regarding biomarkers that was obtained at one or more times, screening tests, or patient visits. For example, islet autoantibody screening tests may be performed for a two-year old child with a family history or genetic risk for Type 1 diabetes. Additional screening tests may be ordered for the child that are to be repeated every six months. The results of the one or more screening tests for the new patient may be input into the disease monitoring system.

In some embodiments, the processor may identify a risk for the new user developing the condition. In some embodiments, determining the risk for the new patient may involve determining where the patient's data (e.g., biomarkers) falls on a previously identified disease progression trajectory. In some embodiments, the new patient's endotype may be related to the patient's age, sex, or baseline characteristics (e.g., age, family medical history, human leukocyte antigen characteristics). In some embodiments, the combination of the new patient's disease progression trajectory and endotype may be used to determine the risk. In some embodiments, the risk for individuals to develop a disease may be calculated using risk calculation methods involving linear regression with regularization and using available research data. In some embodiments, the risk calculation may be modeled/plotted for each disease progression trajectory for each endotype. In some embodiments, the risk assessment may be a calculation of the odds that the new patient may develop the disease by a certain age. In some embodiments, multiple risk calculations may be modeled/plotted for each disease progression trajectory for each endotype, where the multiple risk calculations assess the odds that a patient may develop the disease by different ages.

Continuing the previous example, the disease monitoring system may output that the new patient belongs to disease trajectory X. The disease monitoring system may output that the child's endotype is Y-2-3, where Y-2 reflects the child's age and 3 is a sub-type of the endotype. The risk for the child of developing Type 1 diabetes by age 10 may be 63%, and the risk of developing Type 1 diabetes by age 15 may be 80%.

In some embodiments, the processor may analyze the risk for the new user. In some embodiments, the processor may indicate a suitable remediation group for the new user. In some embodiments, the suitable remediation group may be based on a risk threshold being exceeded. For example, the processor may identify that the risk for the child of developing Type 1 diabetes by age ten is 85%. In some embodiments, the output of the risk assessment (e.g., score or percentage or quantitative value) may be compared to a threshold (e.g., 80%) to determine that the risk level for this child is high. In some embodiments, the processor may determine that a suitable remediation group for this child is group of individuals that are being placed on a clinical trial for a new treatment for Type 1 diabetes.

As another example, a second user with a risk level of 63% may be grouped into a medium risk group based on exceeding a 50% threshold. The remediation measure recommended for the second user may be continued monitoring and screening for Type 1 diabetes. A third user with a risk level of 30% may be grouped into a low risk group based on exceeding a 15% threshold. The remediation measure recommended for the remediation group of the third user may be providing information regarding lifestyle interventions (e.g., drinking more water, taking fewer simple sugars) to the family of the child. In some embodiments, the suitable remediation group may match the patient to a clinical trial, treatment arm, or hospital resource utilization category.

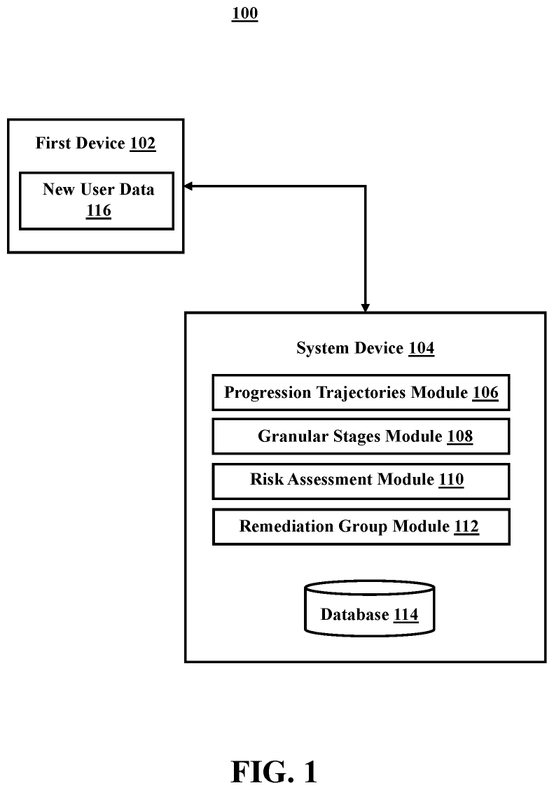

Referring now to FIG. 1, a block diagram of a system 100 for individualized disease trajectory predictions is illustrated. System 100 includes a first device 102 and a system device 104. The system device 104 is configured to be in communication with first device 102. The system device 104 includes a progression trajectories module 106, a granular stages module 108, a risk assessment module 110, and a remediation group module 112. The database 114 stores the data associated with one or more users regarding biomarkers for a condition and the new user data. In some embodiments, the first device 102 and the system device 104 may be any devices that contain a processor configured to perform one or more of the functions or steps described in this disclosure.

In some embodiments, the progression trajectories module 106 determines one or more progression trajectories from the data associated with one or more users regarding biomarkers for a condition. In some embodiments, the progression trajectories may be determined using disease progression modeling, including using Hidden Markov Models. The granular stages module 108 identifies one or more granular stages associated with the progression modeling. The risk assessment module 110 generates a risk assessment for development of the condition. In some embodiments, the risk assessment includes a calculation of the probability of development of a medical condition by a certain age based on the granular stage and progression trajectory of an individual.

In some embodiments, the system device 104 may receive new user data 116 regarding biomarkers of a new user related to the condition from first device 102 The system device 104 may use the risk assessment module 110 to identify a risk for the new user developing the condition. In some embodiments, the remediation group module 112 may analyze the risk assessment for the new user and indicate a suitable remediation group for the user. In some embodiments, the suitable remediation group is suggested (e.g., to the user of the first device 102), at least in part, based on a risk threshold being exceeded.

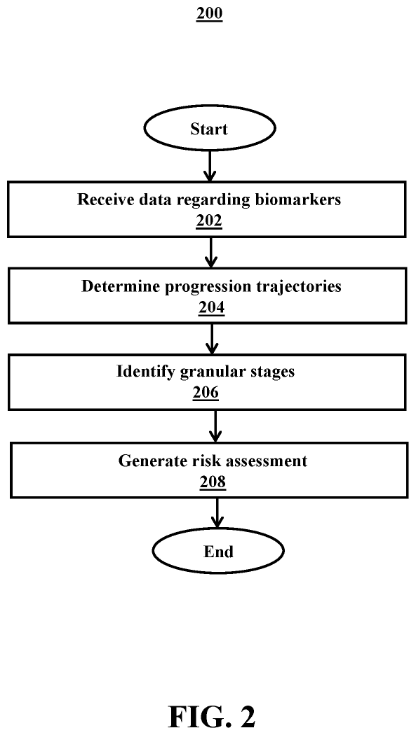

Referring now to FIG. 2, illustrated is a flowchart of an exemplary method 200 for individualized disease trajectory predictions, in accordance with embodiments of the present disclosure. In some embodiments, a processor of a system may perform the operations of the method 200. In some embodiments, method 200 begins at operation 202. At operation 202, the processor receives data associated with one or more users regarding biomarkers for a condition. In some embodiments, method 200 proceeds to operation 204, where the processor determines one or more progression trajectories from the data using progression modeling. In some embodiments, method 200 proceeds to operation 206. At operation 206, the processor identifies one or more granular stages associated with the one or more progression trajectories. In some embodiments, method 200 proceeds to operation 208. At operation 208, the processor generates a risk assessment for development of the condition associated with the one or more progression trajectories and the one or more granular stages.

As discussed in more detail herein, it is contemplated that some or all of the operations of the method 200 may be performed in alternative orders or may not be performed at all; furthermore, multiple operations may occur at the same time or as an internal part of a larger process.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

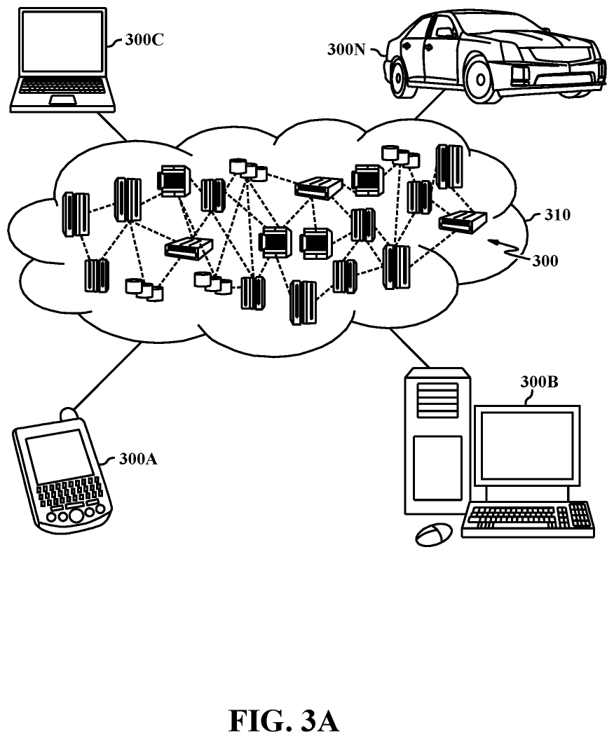

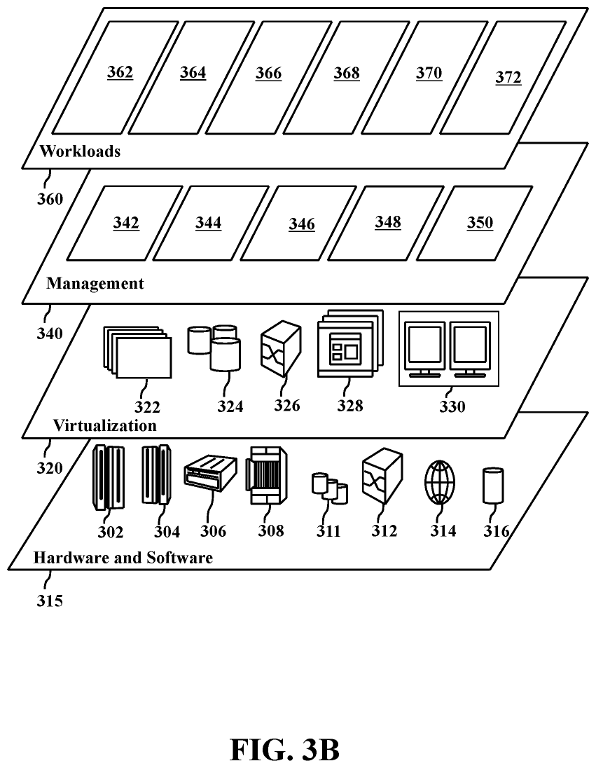

FIG. 3A, illustrated is a cloud computing environment 310 is depicted. As shown, cloud computing environment 310 includes one or more cloud computing nodes 300 with which local computing devices used by cloud consumers, such as, for example, personal digital assistant (PDA) or cellular telephone 300A, desktop computer 300B, laptop computer 300C, and/or automobile computer system 300N may communicate. Nodes 300 may communicate with one another. They may be grouped (not shown) physically or virtually, in one or more networks, such as Private, Community, Public, or Hybrid clouds as described hereinabove, or a combination thereof.

This allows cloud computing environment 310 to offer infrastructure, platforms and/or software as services for which a cloud consumer does not need to maintain resources on a local computing device. It is understood that the types of computing devices 300A-N shown in FIG. 3A are intended to be illustrative only and that computing nodes 300 and cloud computing environment 310 can communicate with any type of computerized device over any type of network and/or network addressable connection (e.g., using a web browser).

FIG. 3B, illustrated is a set of functional abstraction layers provided by cloud computing environment 310 (FIG. 3A) is shown. It should be understood in advance that the components, layers, and functions shown in FIG. 3B are intended to be illustrative only and embodiments of the disclosure are not limited thereto. As depicted below, the following layers and corresponding functions are provided.

Hardware and software layer 315 includes hardware and software components. Examples of hardware components include: mainframes 302; RISC (Reduced Instruction Set Computer) architecture based servers 304; servers 306; blade servers 308; storage devices 311; and networks and networking components 312. In some embodiments, software components include network application server software 314 and database software 316.

Virtualization layer 320 provides an abstraction layer from which the following examples of virtual entities may be provided: virtual servers 322; virtual storage 324; virtual networks 326, including virtual private networks; virtual applications and operating systems 328; and virtual clients 330.

In one example, management layer 340 may provide the functions described below. Resource provisioning 342 provides dynamic procurement of computing resources and other resources that are utilized to perform tasks within the cloud computing environment. Metering and Pricing 344 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 346 provides access to the cloud computing environment for consumers and system administrators. Service level management 348 provides cloud computing resource allocation and management such that required service levels are met. Service Level Agreement (SLA) planning and fulfillment 350 provide pre-arrangement for, and procurement of, cloud computing resources for which a future requirement is anticipated in accordance with an SLA.

Workloads layer 360 provides examples of functionality for which the cloud computing environment may be utilized. Examples of workloads and functions which may be provided from this layer include: mapping and navigation 362; software development and lifecycle management 364; virtual classroom education delivery 366; data analytics processing 368; transaction processing 370; and individualized disease trajectory predictions 372.

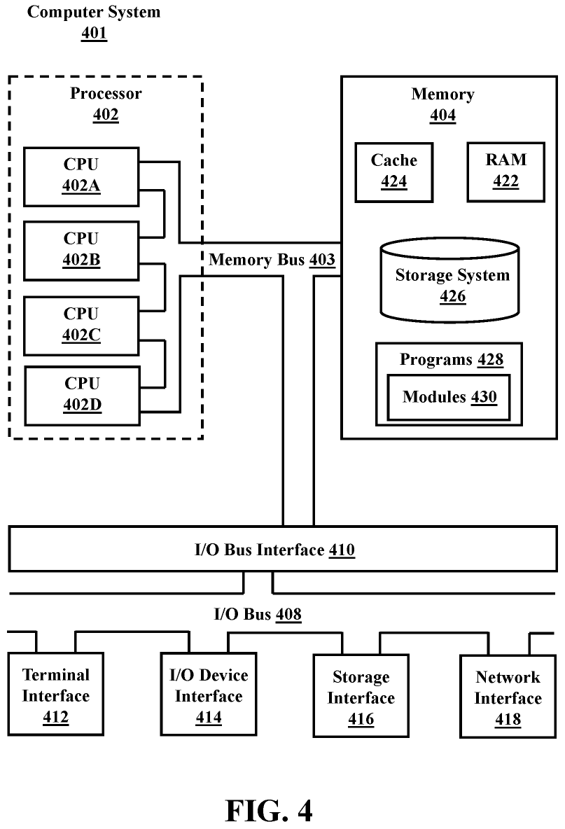

FIG. 4, illustrated is a high-level block diagram of an example computer system 401 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 401 may comprise one or more CPUs 402, a memory subsystem 404, a terminal interface 412, a storage interface 416, an I/O (Input/Output) device interface 414, and a network interface 418, all of which may be communicatively coupled, directly or indirectly, for inter-component communication via a memory bus 403, an I/O bus 408, and an I/O bus interface unit 410.

The computer system 401 may contain one or more general-purpose programmable central processing units (CPUs) 402A, 402B, 402C, and 402D, herein generically referred to as the CPU 402. In some embodiments, the computer system 401 may contain multiple processors typical of a relatively large system; however, in other embodiments the computer system 401 may alternatively be a single CPU system. Each CPU 402 may execute instructions stored in the memory subsystem 404 and may include one or more levels of on-board cache.

System memory 404 may include computer system readable media in the form of volatile memory, such as random access memory (RAM) 422 or cache memory 424. Computer system 401 may further include other removable/non-removable, volatile/non-volatile computer system storage media. By way of example only, storage system 426 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 404 can include flash memory, e.g., a flash memory stick drive or a flash drive. Memory devices can be connected to memory bus 403 by one or more data media interfaces. The memory 404 may include at least one program product having a set (e.g., at least one) of program modules that are configured to carry out the functions of various embodiments.

One or more programs/utilities 428, each having at least one set of program modules 430 may be stored in memory 404. The programs/utilities 428 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 428 and/or program modules 430 generally perform the functions or methodologies of various embodiments.

Although the memory bus 403 is shown in FIG. 4 as a single bus structure providing a direct communication path among the CPUs 402, the memory subsystem 404, and the I/O bus interface 410, the memory bus 403 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 410 and the I/O bus 408 are shown as single respective units, the computer system 401 may, in some embodiments, contain multiple I/O bus interface units 410, multiple I/O buses 408, or both. Further, while multiple I/O interface units are shown, which separate the I/O bus 408 from various communications paths running to the various I/O devices, in other embodiments some or all of the I/O devices may be connected directly to one or more system I/O buses.

In some embodiments, the computer system 401 may be a multi-user mainframe computer system, a single-user system, or a server computer or similar device that has little or no direct user interface, but receives requests from other computer systems (clients). Further, in some embodiments, the computer system 401 may be implemented as a desktop computer, portable computer, laptop or notebook computer, tablet computer, pocket computer, telephone, smartphone, network switches or routers, or any other appropriate type of electronic device.

It is noted that FIG. 4 is intended to depict the representative major components of an exemplary computer system 401. In some embodiments, however, individual components may have greater or lesser complexity than as represented in FIG. 4, components other than or in addition to those shown in FIG. 4 may be present, and the number, type, and configuration of such components may vary.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

What is claimed is:

1. A computer-implemented method, the method comprising:
receiving, by a processor, data associated with one or more users regarding biomarkers for a condition;
determining one or more progression trajectories from the data using progression modeling;
identifying one or more granular stages associated with the one or more progression trajectories; and
generating a risk assessment for development of the condition associated with the one or more progression trajectories and the one or more granular stages.

2. The method of claim 1, further comprising:
receiving new user data regarding biomarkers for the condition; and
identifying a risk for the new user developing the condition.

3. The method of claim 2, further comprising:
analyzing the risk assessment; and
indicating a suitable remediation group for the user.

4. The method of claim 2, wherein the suitable remediation group is based on a risk threshold being exceeded.

5. The method of claim 1, wherein the one or more progression trajectories are determined using Hidden Markov Modeling.

6. The method of claim 1, wherein the one or more progression trajectories are determined by a visual analytics system.

7. A system comprising:
a memory; and
a processor in communication with the memory, the processor being configured to perform operations comprising:
receiving data associated with one or more users regarding biomarkers for a condition;
determining one or more progression trajectories from the data using progression modeling;
identifying one or more granular stages associated with the one or more progression trajectories; and
generating a risk assessment for development of the condition associated with the one or more progression trajectories and the one or more granular stages.

8. The system of claim 7, the processor being further configured to perform operations comprising:
receiving new user data regarding biomarkers for the condition; and
identifying a risk for the new user developing the condition.

9. The system of claim 8, the processor being further configured to perform operations comprising:
analyzing the risk for the new user; and
indicating a suitable remediation group for the new user.

10. The system of claim 8, wherein the suitable remediation group is based on a risk threshold being exceeded.

11. The system of claim 7, wherein the one or more progression trajectories are determined using Hidden Markov Modeling.

12. The system of claim 7, wherein the one or more progression trajectories are determined by a visual analytics system.

13. A computer program product comprising a computer readable storage medium having program instructions embodied therewith, the program instructions executable by a processor to cause the processor to perform operations, the operations comprising:
receiving data associated with one or more users regarding biomarkers for a condition;
determining one or more progression trajectories from the data using progression modeling;
identifying one or more granular stages associated with the one or more progression trajectories; and
generating a risk assessment for development of the condition associated with the one or more progression trajectories and the one or more granular stages.

14. The computer program product of claim 13, the processor being further configured to perform operations comprising:
receiving new user data regarding biomarkers for the condition; and
identifying a risk for the new user developing the condition.

15. The computer program product of claim 14, the processor being further configured to perform operations comprising:
analyzing the risk for the new user; and
indicating a suitable remediation group for the new user.

16. The computer program product of claim 14, wherein the suitable remediation group is based on a risk threshold being exceeded.

17. The computer program product of claim 13, wherein the one or more progression trajectories are determined using Hidden Markov Modeling.

18. The computer program product of claim 13, wherein the one or more progression trajectories are determined by a visual analytics system.
